(19) United States
(12) Patent Application Publication
(10) Pub. No.: US 2026/0345914 A1
(43) Pub. Date: Nov. 5, 2026

(54) DOSING SYSTEMS AND METHODS

(71) Applicant: Nemera Insight Chicago, Chicago, IL (US)

(72) Inventor: Antonio J. BELTON, Richton Park, IL (US)

(21) Appl. No.: 19/196,895

(22) Filed: May 2, 2025

(51) Int. Cl. A61M 5/315

(52) U.S. Cl. CPC A61M 5/31528; A61M 5/3158; A61M 5/31591

(57) ABSTRACT

Systems and methods are disclosed for controlling a dose of medication from a syringe. The dosing system includes a housing configured to be attached to the syringe, a first stop surface and a second stop surface, a button extending from the housing and configured to be actuated by a user, and a drive rod extending at least partially through each of the housing and the syringe. A distal end of the drive rod is configured to engage a plunger stopper within the syringe and the proximal end of the drive rod is configured to be engaged by the button when the button is pressed by the user. Upon the user actuating the button a first time, the drive rod moves distally until it engages the first stop surface whereupon the drive rod rotates. Upon the user actuating the button a second time, the rotated drive rod is able to move further distally until it engages the second stop surface.

### FIELD OF THE INVENTION

The present disclosure relates to dosing systems and methods, including without limitation dosing systems and methods that employ a push button to selectively prime and eject a precise dose of medication, particularly suitable for very small doses of medication.

### BACKGROUND

A conventional syringe typically includes a syringe body defining a volume that receives therein a fluid to be injected, a needle attached to a distal end of the syringe body, a stopper intended to push the fluid towards the needle along an injection direction, and a plunger rod intended to be pressed by a user to push the stopper in the injection direction. It should be noted that, for the purposes of this disclosure, the term “distal” refers to the direction or position towards the needle tip, e.g., in the injection direction, while the term “proximal” refers to the direction or position opposite from distal, e.g., in this context, away from the needle tip and towards the user actuator/button.

Precise dosing of a syringe is important for many applications, for example and without limitation, ophthalmic injections and in particular intravitreal injections. The eye being a small and closed environment, an injection which is not well controlled could lead to an unwanted intraocular pressure. Moreover, injection volumes could be as small as 0.03 ml to 0.1 ml, which also require a precise dosage.

The use of pre-filled syringes (PFS) is preferred for many applications, for example intravitreal injections, because it decreases the risk of microbial and particle contamination of the fluid to be injected as compared to syringes that require a filling step during which the fluid to be injected is withdrawn from a separated vial via the needle. Moreover, the use of PFS reduces the injection preparation time.

Existing PFS are usually designed for injection volumes higher or equal to 0.5 ml. They are typically provided with gradations on the syringe body with which the user may visually align a part of the stopper to set the dose to be injected. This kind of dosing system depends on the user interpretation of this alignment, which could lead to a lack of precision. This risk is particularly important when a syringe that is designed for injection volumes of 0.5 ml to 1.0 ml is instead used for a lower injection volume.

Thus, when a larger volume of medication may be present in a conventional syringe prior to the syringe's use—e.g., due to the difficulty in loading very small amounts in the syringe during manufacturing, the desirability of making sure that there is sufficient medication present in the syringe at the time of use so as to avoid under-dosing, the need to account for air in the syringe, etc.—and the correct dose of medication desired to be dispensed may be a lesser amount, the unintended dispensing of the full amount of medication in the syringe may, as indicated above, result in overdosing.

As mentioned above, current methods, particularly when using pre-filled syringes, often depend on the syringes including visual markers. A user may employ these visual markers, prior to actual use of the syringe to dispense the medication, to move the components of the syringe relative to each other until the visual markers are visually aligned. The visually alignment of these visual markers may indicate to the user that the air in the syringe has been removed and/or that the components of the syringe are in the correct position relative to each other such that, upon the user actuating the syringe, the correct dosage of medication is dispensed.

However, and as alluded to above, such prior art systems and methods are prone to human error, e.g., during the preparation and administration of syringe-based injections. Such human errors can lead to inaccuracies in the dose being set and ultimately delivered. For example, these errors may arise from misinterpreting syringe markers, improper purging of air bubbles, or deviations in technique. Such inaccuracies can result in underdosing or overdosing, potentially compromising the effectiveness of the treatment or causing adverse effects, thereby posing significant risks to patient safety and treatment outcomes.

Thus, there is a need for systems and methods that ensure accurate delivery of medication via a syringe, and specifically accurate delivery of a precise dose of a small volume of medication. Thus, a goal of this disclosure is to provide a precise dosing system compatible with the use of PFSs and which allows user-independent dosing.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

As mentioned above and as will be apparent from the various embodiments set forth below, the dosing system described herein may address one or more of these various challenges. For example, in various embodiments, there are disclosed systems and methods for controlling a dose of medication from a syringe. The dosing system may include a housing configured to be attached to the syringe, a first stop surface and a second stop surface, a button extending from the housing and configured to be actuated by a user, and a drive rod extending at least partially through each of the housing and the syringe. A distal end of the drive rod may be configured to engage a plunger stopper within the syringe and the proximal end of the drive rod may be configured to be engaged by the button when the button is pressed by the user. Upon the user actuating the button a first time, the drive rod moves distally until it engages the first stop surface, and which point the drive rod may rotate a first time. This may constitute priming the syringe or it may comprise ejecting a dose of medication from the syringe, depending on the function desired. Upon the user actuating the button a second time, the already-rotated drive rod may continue to move distally past the first stop surface until it engages the second stop surface thereby ejecting a dose of medication from the syringe. During the first button actuation, the drive rod may be blocked in rotation during a first translation phase, before it engages the first stop surface. During the second button actuation, the drive rod may be blocked in rotation during a second translation phase, before it engages the second stop surface.

In some embodiments, the housing may comprise a body configured to be attached to the syringe and a collar configured to be attached to the body. The button may extend from the collar.

In some embodiments, the first stop surface may be a first shoulder located on an inner surface of the housing, in particular an inner surface of the body, the first shoulder configured to be contacted by the drive rod when the user actuates the button the first time. Also, the second stop surface may be a second shoulder located on the inner surface of the housing, in particular an inner surface of the body, the second shoulder located distally relative to the first shoulder and configured to be contacted by the drive rod when the user actuates the button the second time. The second shoulder is angularly offset compared to the first shoulder.

The drive rod may comprise a radial protrusion. The protrusion of the drive rod may be configured to cooperate with a slot of the housing to block the drive rod in rotation during the first translation phase.

The dosing system may also include a first anti-return surface. The first anti-return surface is configured to limit the drive rod proximal movement after the user releases the button the first time. The first anti-return surface may be configured to be contacted by the drive rod when the user, after having actuated the button the first time, releases the button a first time. The first anti-return surface may be a first ramp located on an inner surface of the housing, in particular an inner surface of the collar, the first ramp configured to be contacted by the radial protrusion of the drive rod when the user, after having actuated the button the first time, releases the button a first time. In some embodiments, the dosing system comprises a plurality of first anti-return surfaces, for example between two and four, and the drive rod comprises a plurality of protrusions, for example between two and four, each protrusion being able to cooperate with a corresponding anti-return surface. The drive rod may include a plurality of proximally-extending teeth, the drive rod being configured to rotate when the plurality of proximally-extending teeth engage distally-extending teeth of the button. The plurality of proximally-extending teeth of the drive rod may define a crown. The plurality of distally-extending teeth of the button may define a crown. The plurality of proximally-extending teeth of the drive rod may be able to engage the distally-extending teeth of the button during a final phase of the user actuation of the button for the first time, when the drive rod is no longer blocked in rotation. The rotation of the drive rod may be initiated by the engagement of the plurality of proximally-extending teeth of the drive rod with the distally-extending teeth of the button. This engagement may be allowed during the final phase of the user actuation of the button for the first time and which occurs after the first translation phase, when the protrusion of the drive rod is free from the slot of the housing. At the end of the user actuation of the button for the first time, the radial protrusion of the drive rod is facing the first ramp and positioned distally relative to the first ramp. Upon the user releasing the button the first time, the radial protrusion of the drive rod slide along the first ramp. The first ramp may be angled proximally such that, upon the user releasing the button the first time, the drive rod moves proximally, while the protrusion of the drive rod slides along the first ramp, until the protrusion engages a first wall. In embodiments, when the protrusion engages the first wall, the syringe may be in a primed position. The protrusion may cooperate with the first wall to block the drive rod in rotation during the second translation phase.

The dosing system may also include a second anti-return surface. The second anti-return surface is configured to limit the drive rod proximal movement after the user releases the button the second time. The second anti-return surface may be configured to be contacted by the drive rod when the user, after having actuated the button the second time, releases the button a second time. The second anti-return surface may be a second ramp located on an inner surface of the collar, the second ramp configured to be contacted by the radial protrusion of the drive rod when the user, after having actuated the button the second time, releases the button a second time. The drive rod may be configured to further rotate by the plurality of distally-extending teeth of the button engaging the plurality of proximally-extending teeth of the drive rod. The plurality of proximally-extending teeth of the drive rod may be able to engage the distally-extending teeth of the button during a final phase of the user actuation of the button for the second time, when the drive rod is no longer blocked in rotation. The further rotation of the drive rod may be initiated by the engagement of the plurality of proximally-extending teeth of the drive rod with the distally-extending teeth of the button. This engagement may be allowed during the final phase of the user actuation of the button for the second time and which occurs after the second translation phase, when the protrusion of the drive rod is free from the first wall. At the end of the user actuation of the button for the second time, the radial protrusion of the drive rod is facing the second ramp and positioned distally relative to the second ramp. Upon the user releasing the button the second time, the protrusion of the drive rod slides along the second ramp. The second ramp may be angled proximally such that, upon the user releasing the button the second time, the drive rod moves proximally, while the protrusion is sliding along the second ramp, until the protrusion engages a second wall. In embodiments, when the protrusion engages the second wall, the dosing system may be in a locked position.

In embodiments, the dose system may also include a biasing member configured to move the drive rod proximally in the absence of force exerted by the user on the button during either the first and second actuations of the button. The biasing member may be a spring. The button may have a protuberance cooperating with a slot of the housing, in particular a slot of the collar, so as to enable the button to move longitudinally and prevent rotation of the button when the user actuates the button. The slot of the housing which cooperates with the protuberance of the button and the slot of the housing which cooperates with the protrusion of the drive rod during the translation phase may be a single slot. Also, the system may include an indicator providing information to the user on the position of the drive rod. For example, the indicator may be a tactile indicator that provides the user with tactile feedback at least one of when the drive rod has moved distally and when the drive rod has moved further distally, for example when the syringe has been primed and when the syringe has ejected the medication. Additionally or alternatively, the indicator may be an audible indicator that provides an audible indication to the user at least one of when the drive rod has moved distally and when the drive rod has moved further distally, for example when the syringe has been primed and when the syringe has ejected the medication. Additionally or alternatively, the indicator may be a visual indicator that provides a visual indication to the user at least one of when the drive rod has moved distally and when the drive rod has moved further distally, for example when the syringe has been primed and when the syringe has ejected the medication.

The dosing system may also include a retainer, and a flange of the syringe may be configured to be maintained, e.g., compressed, between the retainer and the housing, in particular between the retainer and the body of the housing. In embodiments, the syringe is a pre-filled syringe.

As set forth above, in embodiments, the user actuating the button the first time may correspond to a priming step, and the user actuating the button the second time corresponds to a dosing step, or the user actuating the button the first and second times may both correspond to a dosing step. Indeed, in various embodiments, the user may actuate the button a third, or more, times, each such time corresponding to another dosing step. The priming step is a step for readying a dose of medication to be ejected by the syringe. The priming step may allow purging air from the syringe. The dosing step is a step for ejecting a dose of medication from the syringe, for example for dispensing the dose of medication to a patient.

Also provided herein is, according to various embodiments, a method for dispending a dose of medication from a syringe. In various embodiments, the method includes the step of providing a dosing system that includes a housing configured to be attached to the syringe, optionally the housing comprises a body configured to be attached to the syringe and a collar configured to be attached to the body (the body and collar are separate components), a first stop surface and a second stop surface, a button extending from the housing and configured to be actuated by a user, and a drive rod extending at least partially through each of the housing and the syringe. The method may also include the step of priming the syringe or ejecting a first dose of medication from the syringe by pressing the button a first time such that the drive rod moves distally until it engages the first stop surface and releasing the button a first time such that the drive rod moves proximally until it engages a first anti-return surface. The method may also include the step of ejecting a first or second dose of medication from the syringe by pressing the button a second time so as to move the drive rod still further distally until it engages the second stop surface of the collar. The method may also include the step of locking the syringe by releasing the button a second time such that the drive rod again moves proximally until it engages a second anti-return surface. In embodiments, the drive rod rotates upon each one of the first and second pressing steps of the button.

In such embodiments, the first stop surface may be a first shoulder located on an inner surface of the housing, in particular an inner surface of the body, and the first pressing step may include the first shoulder contacting the drive rod. The second stop surface may be a second shoulder located on the inner surface of the housing, in particular an inner surface of the body, and the second pressing step may include the drive rod contacting the second shoulder. The first anti-return surface may be a first ramp located on an inner surface of the housing, in particular an inner surface of the collar, the first releasing step including the first ramp contacting the drive rod. The drive rod may include a plurality of proximally-extending teeth, the method further comprising the step of rotating the drive rod by such plurality of proximally-extending teeth of the drive rod engaging a plurality of distally-extending teeth of the button. The radial protrusion of the drive rod engage the first ramp during the first releasing step. The first releasing step may include the drive rod moving proximally with the protrusion sliding along the first ramp until the protrusion engage a first wall. The second anti-return surface may be a second ramp located on an inner surface of the housing, in particular an inner surface of the collar, the second releasing step including the protrusion of the drive rod contacting the second ramp.

In embodiments, the method may also include the step of further rotating the drive rod by the plurality of proximally-extending teeth of the drive rod engaging the plurality of distally-extending teeth of the button. The radial protrusion on the drive rod may engage the second ramp during the second releasing step. The method may also include a biasing member configured to move the drive rod proximally in the absence of force exerted by the user on the button during either the first and second pressings of the button. The biasing member may be a spring. The method may also include the step of providing to the user at least one of a visual, a tactile or an audible indication on the position of the drive rod, for example after one or both of the button releasing steps.

In various embodiments, the dosing system may also include a retainer, and the method may also include the step of maintaining a flange of the syringe between the retainer and the housing, in particular the body of the housing. The syringe may be a pre-filled syringe.

In still further embodiments there is provides a dosing system for a syringe, the dosing system comprising: a housing configured to be attached to the syringe; a first stop surface and a first stop wall; a button extending from the housing and configured to be actuated by a user; and a rotatable drive rod extending at least partially through the housing and the syringe, a distal end of the drive rod configured to engage a plunger stopper within the syringe and the proximal end of the drive rod configured to be engaged by the button when the button is pressed by the user. Upon the user pressing the button a first time, the drive rod may move distally until it engages the first stop surface. Upon the user releasing the button a first time, the drive rod may rotate a first time until it contacts the first stop wall. The user pressing the button the first time may correspond to either a priming step for readying a dose of medication to be ejected by the syringe or a dosing step for ejecting the dose of medication from the syringe.

The dosing system may comprise a second stop surface and be configured such that, when the user presses the button a second time, the rotated drive rod is free to move distally past the first stop surface until it engages the second stop surface. The user pressing the button the second time may correspond to a dosing step for ejecting a first dose of medication. The dosing system may be configured such that, when the user releases the button a second time, the drive rod may rotate a second time until it contacts a second stop wall. The step of releasing the button a second time may correspond to a locking step. The system may be configured such that, when the user presses the button a third time, the dosing system may eject a second dose of medication.

The dosing system may be for a medical injection, in particular a parenteral injection, in particular an intraocular injection, in particular an intravitreal injection and/or a pediatric injection. The dosing system is be configured to eject a dose of medication from the syringe. The dose of medication may be of a volume comprised between 0.01 ml and 0.4 ml, in particular between 0.03 ml and 0.1 ml. The dosing system may be configured to eject a medication that may comprise for example Bevacizumab, Ranibizumab, Brolucizumab, Faricimab, Aflibercept, Pegcetacoplan, Avacincaptad pegol, Triamcinolone acetonide, Ganciclovir, Clindamycine, Foscarnet, Fomivirsen, Methotrexate, Vancomycine, Ceftazidime, Amikacine, Amphotéricine, Voriconazole, Pegaptanib, or their salts, solvates, hydrates or isomers or any medical formulation adapted to intraocular injection.

The dosing system as set forth herein, in various embodiments thereof, may provide various advantages over conventional syringe systems for controlled drug delivery applications. For example, the dosingdosing system as set forth herein may provide enhanced precision as compared to conventional syringe systems. As an example of such enhanced precision, the dosing system as set forth herein, according to various embodiments, may allow for incremental, controlled adjustments of the plunger stopper, enabling highly accurate dose setting and delivery without relying on visual estimation or manual dexterity. This is particularly beneficial for delivering small volumes.

The dosing system as set forth herein, in various embodiments thereof, may provide additional advantages over conventional syringe systems for controlled drug delivery applications. For example, the dosing system as set forth herein may provide a more user-friendly operation as compared to conventional syringe systems. As an example of such more user-friendly operation, the dosing system as set forth herein, according to various embodiments, may provide feedback to the user. Such feedback may provide clear confirmation of each adjustment, reducing ambiguity during dose preparation. This intuitive interface makes the process simpler and more consistent, especially for users with limited training or physical dexterity.

Still other advantages may be provided by the dosing system as set forth herein, in various embodiments thereof, over conventional syringe systems for controlled drug delivery applications. For example, the dosing system as set forth herein may provide a reduced risk of errors as compared to conventional syringe systems. Traditional plungers typically require careful alignment and manual stopping to avoid overdosing or underdosing. In contrast, the dosing system as set forth herein, according to various embodiments, inherently restricts movement to pre-calibrated increments, minimizing the potential for human error.

Another advantage that may be provided by the dosing system as set forth herein, in various embodiments thereof, over conventional syringe systems for controlled drug delivery applications are the built-in safety features thereof. For example, the dosing system as set forth herein, according to various embodiments, can be designed to limit plunger stopper travel. Limiting the plunger stopper travel may help prevent over-compression of the plunger stopper and eliminating the risk of a vacuum effect at the end of delivery. This ensures the integrity of the dose and prevents issues like drug leakage or incomplete delivery.

Yet another advantage that may be provided by the dosing system as set forth herein, in various embodiments thereof, over conventional syringe systems for controlled drug delivery applications are its versatility and scalability. The dosing system as set forth herein, according to various embodiments, can be adapted to various syringe types, including prefilled and standard syringes, making it suitable for a wide range of applications and scalable for mass production.

### DRAWINGS

FIG. 1A is a perspective assembled view that illustrate a dosing system, in accordance with various embodiments.

FIG. 1B is a side exploded view of the dosing system, in accordance with various embodiments.

FIG. 1C is a perspective view of a collar, in accordance with various embodiments.

FIG. 1D is a side perspective view of a drive rod, in accordance with various embodiments.

FIG. 1E is another angled perspective view of the drive rod of FIG. 1D, in accordance with various embodiments.

FIG. 2A is a side cross-sectional view of the collar, in accordance with various embodiments.

FIG. 2B is an assembled side cross-sectional view of the dosing system in a pre-use position, in accordance with various embodiments.

FIG. 2C is a perspective view of the proximal components of the dosing system in the pre-use position with the collar and the body being shown in phantom, in accordance with various embodiments.

FIG. 3A is an assembled side cross-sectional view of the dosing system in a first partially primed position, in accordance with various embodiments, showing the components thereof in a fully assembled position.

FIG. 3B is a perspective view of the proximal components of the dosing system in second partially primed position, with the collar and the body being shown in phantom, in accordance with various embodiments.

FIG. 4A is an assembled side cross-sectional view of the dosing system, in accordance with various embodiments, showing the components thereof in a fully assembled position and in the position of FIG. 3B.

FIG. 4B is a perspective view of the proximal components of the dosing system in third partially primed position with the collar and the body shown in phantom, in accordance with various embodiments.

FIG. 4C is another perspective view of the proximal components of the dosing system in a fully primed position with the collar and the body shown in phantom, in accordance with various embodiments.

FIG. 5A is an assembled side cross-sectional view of the dosing system in the dosed position, in accordance with various embodiments, showing the components thereof in a fully assembled position.

FIG. 5B is a perspective view of the proximal components of the dosing system in the dosed position with the collar and the body shown in phantom, in accordance with various embodiments.

FIG. 5C is a perspective view of the proximal components of the dosing system in a partially locked position with the collar and the body shown in phantom, in accordance with various embodiments.

FIG. 5D is a perspective view of the proximal components of the dosing system in a fully locked position with the collar and the body shown in phantom, in accordance with various embodiments.

FIG. 6A illustrates a first step of the system's operation by which the user presses the button 11 a first time so as to prime the syringe, in accordance with various embodiments.

FIG. 6B illustrates a second step of the system's operation by which the user positions the distal tip of the syringe 18 relative to a patient, in accordance with various embodiments.

FIG. 6C illustrates a third step of the system's operation, by which the user presses the button 11 a second time so as to dispense medication, in accordance with various embodiments.

FIG. 6D illustrates a fourth step of the system's operation, by which the syringe may be disposed of in a safe manner, in accordance with various embodiments.

### DETAILED DESCRIPTION

Reference will now be made in detail to specific embodiments illustrated in the accompanying drawings. In the following detailed description, numerous specific details are set forth to provide a thorough understanding. However, it will be apparent to one of ordinary skill in the art that embodiments may be practiced without these specific details. In other instances, known methods, procedures and/or components have not been described in detail so as not to unnecessarily obscure aspects of the embodiments.

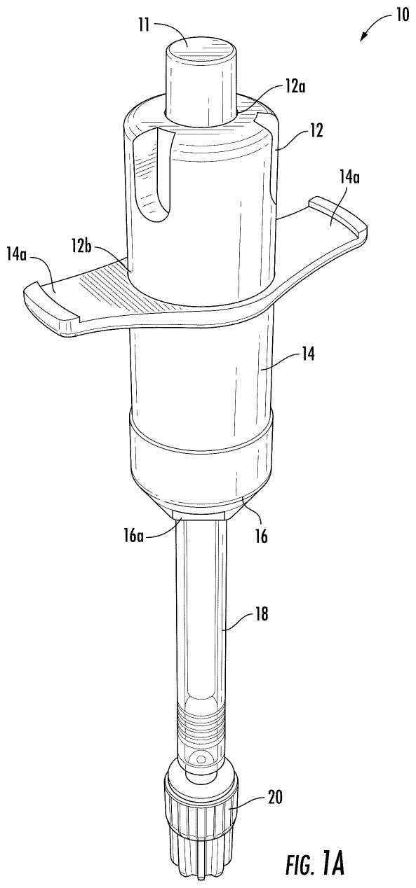

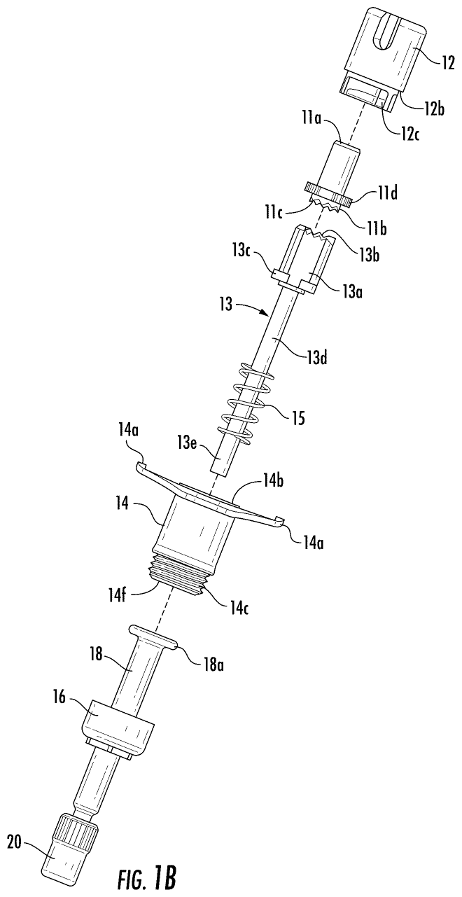

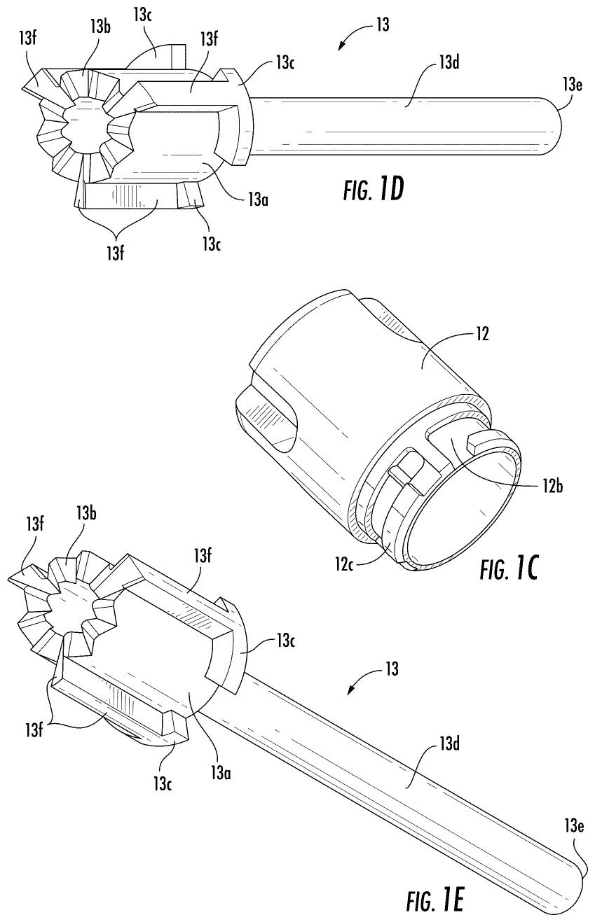

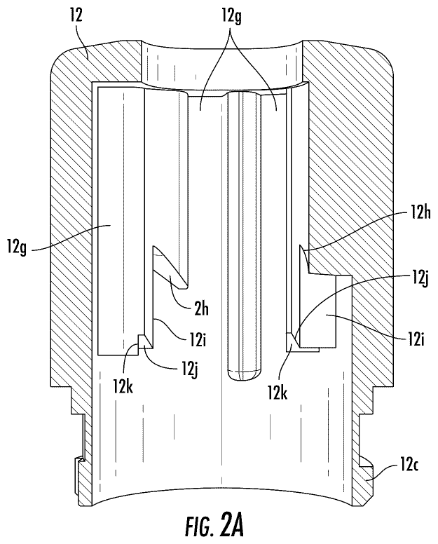

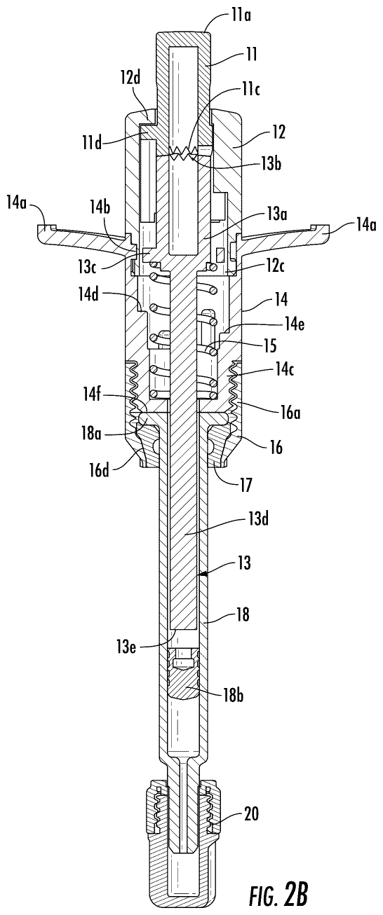

FIG. 1A is a perspective assembled view that illustrate a dosing system 10, in accordance with an embodiment thereof. In the embodiment shown, the system 10 includes a collar 12. Positioned partially within and extending through a central-disposed, proximal opening 12a of the collar 12 is a button 11. Attached to a distal end 12b of the collar 12 is a body 14. The assembly of the collar 12 and the body 14 forms a housing. The body 14 has a pair of oppositely-disposed, radially extending finger tabs 14a. Attached to distal end 14f of the collar 14 is a retainer 16. Extending through a central-disposed, distal opening 16a of the retainer 16 is a syringe body 18. Attached to a distalmost end of the syringe body 18 is syringe safety cap 20. The syringe safety cap 20 is configured to be removed from the syringe body and a needle may be attached to the distalmost end of the syringe body 18.

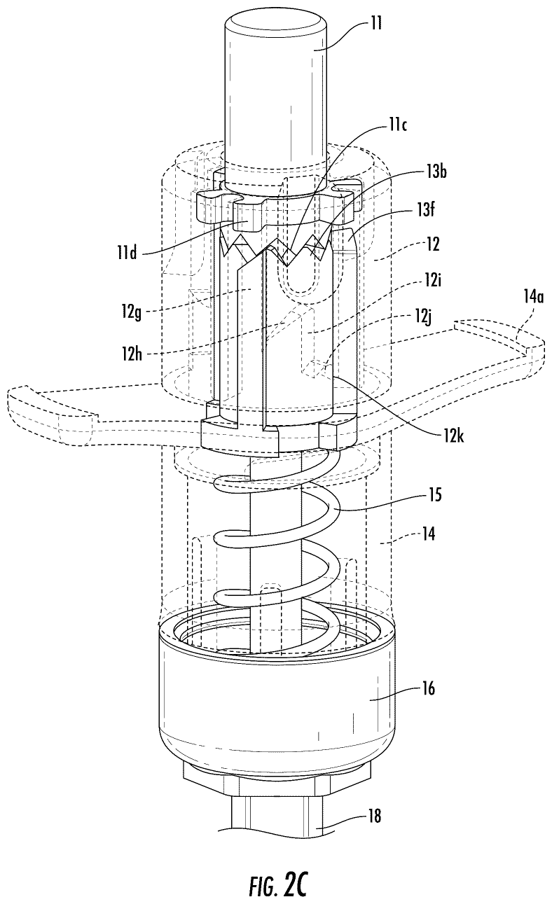

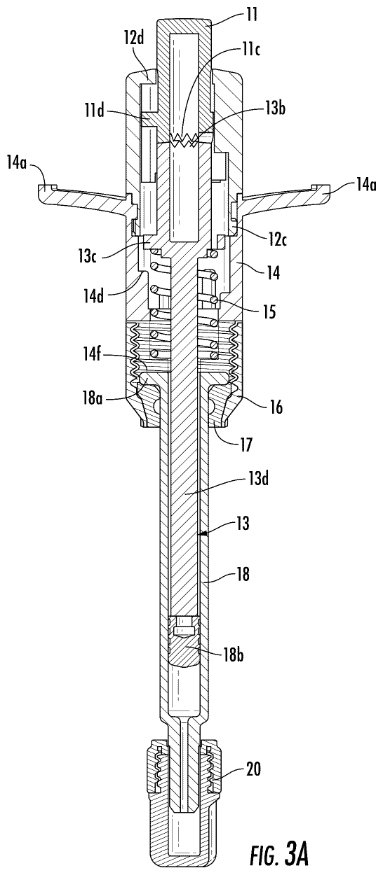

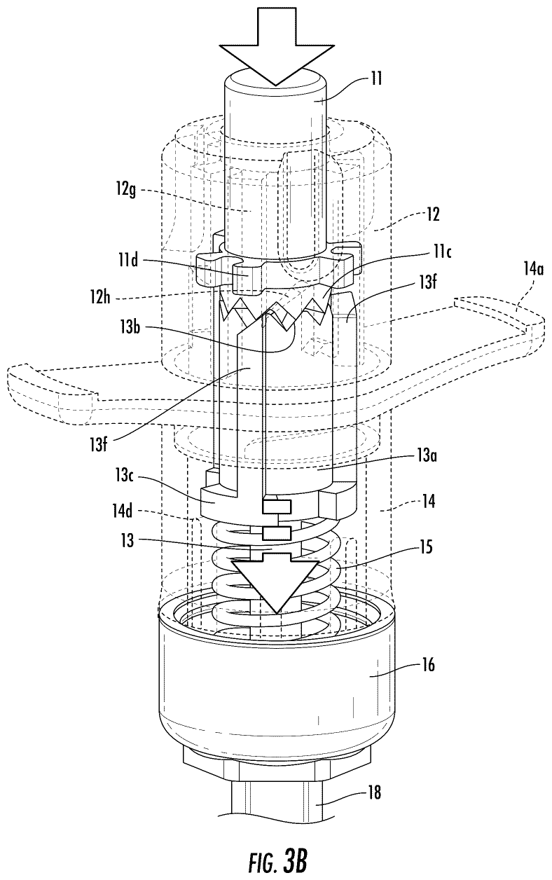

FIG. 1B is a side exploded view of the dosing system 10, in accordance with this embodiment, showing additional details and components thereof. As shown, the collar 12 includes a connection mechanism 12c, which extends from the distal end 12b of the collar 12. The connection mechanism 12c of the collar 12 is connectable to a complementary connection mechanism 14b (hidden in this view but shown in, e.g., FIG. 2B) extending from the proximal end of the body 14, as is explained in greater detail below. It should be noted that the body 14 and the collar 12 of the housing may be separate components, as shown herein, or the two components may instead be incorporated into a single housing component which includes all of the salient features thereof. FIG. 1C is a perspective view of the collar 12, in accordance with this embodiment. Additional details of the interior of the collar 12 are shown in FIG. 2A, which is a side cross-sectional view of the collar and which is described in greater detail below.

FIG. 1B also shows additional features of the button 11. The button 11 forms a cylinder at its proximal end 11a, although other shapes of the proximal end 11a of the button 11 are envisioned. The button 11 also includes at its distal end 11b a plurality of distally-extending teeth 11c. Located just proximal to the distal end 11b of the button are a series of radially-extending protuberances 11d that collectively form a flange-like shape. As set forth above, the button 11 is positioned within the interior of the collar 12 and extends through a central-disposed, proximal opening 12a of the collar 12.

FIG. 1B also shows additional components that are hidden from view in FIG. 1A. For example, FIG. 1B also shows that the system 10 includes a drive rod 13. The drive rod 13 includes at its proximal end a head portion 13a, the proximal-most face of which includes a plurality of proximally-extending teeth 13b configured to engage, e.g., mesh, with the distally-extending teeth 11c of the button 11. In addition, and as best shown in FIGS. 1D and 1E, which are perspective views of the drive rod 13 alone, radially extending from the head portion 13a of the drive rod 13 are protrusions 13f. The protrusions 13f may be, as illustrated in FIGS. 1D and 1E, radial extensions of one or more of the plurality of proximally-extending teeth 13 b. In various embodiments, the protrusions 13f may be radial extensions of an entire tooth of such one or more proximally-extending teeth 13b, or in still other embodiments, the protrusions 13f may be radial extensions of merely a portion of a tooth of such one or more proximally-extending teeth 13b. As illustrated in FIGS. 1D and 1E, in the embodiment shown herein, the protrusions 13f are radial extensions of a half-portion, e.g., one slanted surfaces of the tooth, of three of the plurality of proximally-extending teeth 13b. Extending distally from the head portion 13a of the drive rod 13 is a shaft 13d having a distal tip 13e. The shaft 13d has a transversal cross-section dimension, e.g., diameter in this case, that is lower than a transversal cross-section dimension of the head portion 13a. Disposed around the shaft 13d of the drive rod 13 is a biasing member, in this case a coil spring 15, although other types of springs are contemplated as well. The proximal end of the spring 15 is configured, when the system 10 is fully assembled, to abut the distal face of the head portion 13a of the drive rod 13.

As mentioned above in connection with FIG. 1A, the distal end 12b of the collar 12 is attached to the body 14, such as via a twist-lock arrangement, although other connection mechanisms, e.g., a screw connection, a snap-lock connection, etc. are also contemplated. In addition to a pair of oppositely-disposed finger tabs 14a that extend radially outwardly from the body 14. The body 14 also includes a connection mechanism 14c extending from the distal end 14f of the body 14. In the embodiment shown, the connection mechanism 14c is a screw-type connection, although other types of connection mechanism, e.g., a twist-lock connection, a snap-lock connection, etc. are also contemplated.

The retainer 16 has an internal connection mechanism 16a (hidden in this view of FIG. 1B, but shown in FIG. 2B). The connection mechanism 16a of the retainer 16 is complementary to the connection mechanism 14c extending from the distal end 14f of the body 14 such that the body 14 and the retainer 16 can be selectively connected to each other. In the embodiment shown, the connection mechanism 16a of the retainer 16 is a screw-type connection that matches the screw-type connection of the connection mechanism 14c of the body 14. However, other types of connection mechanisms 16a of the retainer 16 are contemplated, e.g., a twist-lock connection, a snap-lock connection, etc. so long as the connection mechanism 16a of the retainer 16 is complementary to the connection mechanism 14c extending from the distal end 14f of the body 14. As set forth above, the syringe body 18 extends through a central-disposed, distal opening 16a of the retainer 16. A proximal end of the syringe 18 includes a syringe flange 18a. Attached to a distalmost end of the syringe 18 is syringe safety cap 20 which also ensures that medication within the distal end of the syringe 18 is retained therein until desired to be dispensed.

FIG. 2A, as set forth above, is a side cross-sectional view of the collar 12, according to various embodiments. As shown in FIG. 2A, the interior wall of the collar includes a first set of longitudinal slots 12g that extend longitudinally along the inner surface of the collar 12. Intersecting each one of the longitudinal slots 12g, partially along the longitudinal length of the longitudinal slots 12g, are a series of first ramps 12h, for example used in the priming step as set forth below, that extending circumferentially along the inner surface of the collar 12 and that angle slightly proximally relative to the point at which the first ramps 12h intersect their respective longitudinal slots 12g. In addition, at the proximal-most end of each one of the series of first ramps 12h is a longitudinal-extending wall 12i, for example used during the dosing step as set forth below. Each longitudinally-extending wall 12i extends from the first ramp 12h to a respective one of a series of second ramps 12j, again for example used during the dosing step as set forth below. Each one of the second ramps 12j extend circumferentially along the inner surface of the collar 12 and angle slightly proximally relative to the point at which the second ramps 12j intersect their respective longitudinal walls 12i. Finally, at the proximal-most end of each one of the series of second ramps 12j is a longitudinal-extending stop wall 12k. It should be noted that, while embodiment shown includes two ramps, e.g., first ramp 12h and second ramp 12j, embodiments are also contemplated in which the collar 12 includes less than two or more than two ramps depending on the number of steps desired during operation, each such ramp being radially distributed along the inner surface of the collar 12. It should also be noted that, while embodiment shown includes a first ramp 12h that is employed during a priming step and a second ramp 12j that is employed during a dosing step, embodiments are also contemplated in which structures other than ramps could be employed.

FIG. 2B is an assembled side cross-sectional view of the dosing system 10, in accordance with this embodiment, showing the components thereof in a fully assembled position, and showing the dosing system 10 in a pre-use position, e.g., prior to the system being used. As shown, the connection mechanism 12c of the collar 12 is connected to the complementary connection mechanism 14b extending from the proximal end of the body 14. Likewise, the connection mechanism 14c extending from the distal end of the body 14 is connected to the connection mechanism 16a of the retainer 16. Inside of the retainer 16 is a gasket 17, with the syringe flange 18a of the syringe 18 being sandwiched between a distal-most face 14f of the body 14 and the gasket 17. The connection mechanisms 14c and 16a (in this case the complementary screw mechanisms) of the body 14 and the retainer 16, respectively, apply pressure from the distal-most face of the 14f of the body 14 to the syringe flange 18a to the gasket 17 and to an inner distal face 16d of the retainer, thereby causing the gasket 17 to maintain a compression between these components.

FIG. 2B also shows the button 11 in its proximal-most position within interior of the collar 12, with the series of radially-extending protuberances 11d of the button 11 abutting the radially-inwardly extending shoulder 12d of the collar 12. The plurality of distally-extending teeth 11c of the button 11 are offset from the plurality of proximally-extending teeth 13b at the proximal end of the drive rod 13. By applying a proximally-directed force on the drive rod 13, the spring 15 maintains the button 11 and the drive rod 13 in these respective proximal-most positions. In addition, the spring 15 maintains the radially-extending flange 13c on the head 13a of the drive rod 13 in a spaced apart relationship with a first inner shoulder 14d of the body 14. The body 14 also includes a second inner shoulder 14e that is located distally relative to the first inner shoulder 14d and angularly offset relative to the first inner shoulder 14d, the purpose of which will be described in greater detail below. In this pre-use position, the shaft 13d of the drive rod 13 extends partially through the interior of the syringe 18 and may be spaced apart from a syringe plunger stopper 18b that is configured to slide distally through the interior of the syringe 18 upon distal engagement by the distal-most tip 13e of the shaft 13d of the drive rod 13.

FIG. 2C is a perspective view of the proximal components of the dosing system 10 in pre-use position, this time with certain components of the system 10 in phantom so as to better illustrate the interior components therewithin. In this case, the collar 12 and the body 14 are shown in phantom. As shown here, the interior of the collar includes the first set of longitudinal slots 12g that extend longitudinally along the inner surface of the collar 12, and intersecting each one of the longitudinal slots 12g, partially along the longitudinal length of the longitudinal slots 12g, are the series of first ramps 12h that extending circumferentially along the inner surface of the collar 12 and that angle slightly proximally relative to the point at which the first ramps 12h intersect their respective longitudinal slots 12g. In addition, as previously described and shown in connection with FIG. 2A, the proximal-most end of each one of the series of first ramps 12h connects to the longitudinal-extending walls 12i, and the longitudinal-extending walls 12i extends from the first ramp 12h to respective ones of the second ramps 12j. Each one of the second ramps 12j extend circumferentially along the inner surface of the collar 12 and angle slightly proximally relative to the point at which the second ramps 12j intersect their respective longitudinal walls 12i. At the proximal-most end of each one of the series of second ramps 12j is the longitudinal-extending stop wall 12k. In the position shown in FIG. 2C, the radially-extending protuberances 11d of the button 11 are positioned at the proximal-most ends of the longitudinally-extending slots 12g, maintained in this position by the spring 15. In this position, the protrusions 13f of the drive rod 13 extends along the longitudinal slots 12g which prevents a rotation of the drive rod 13.

FIG. 3A is an assembled side cross-sectional view of the dosing system 10, in accordance with this embodiment, showing the components thereof in a fully assembled position, and showing the dosing system 10 in a position at which a user has begun to apply a first longitudinal, distally-directed pressure to the button 11. This first longitudinal, distally-directed pressure to the button 11 functions to help prime the syringe 18 dosing, as will be set forth in additional detail below. In general, priming refers to the step by which the dosing system is placed into a position at which it is prepared to, but does not yet in fact, dispense medication therefrom. Advantageously, and as set forth in additional detail below, the step of priming described herein also refers to the step of purging any air and/or excess medication from the syringe prior to dispensing medication therefrom, so as to eliminate mis-dosing and/or safety concerns related to either air or excess medication being unintentionally dispensed. FIG. 3A shows the system 10 in a partially primed position.

As shown in FIG. 3A, the button 11 has begun to move distally with the series of radially-extending protuberances 11d of the button 11 no longer abutting the radially-inwardly extending shoulder 12d of the collar 12. The plurality of distally-extending teeth 11c of the button 11 remain offset from the plurality of proximally-extending teeth 13b at the proximal end of the drive rod 13. In this position, the force of the spring 15 has been at least partially overcome by the force applied by the user to the button 11, with the radially-extending protuberances 11d of the collar 11 travelling partway along and through the longitudinal slots 12g along the inner surface of the collar 12, the button 11 and the drive rod 13 translating longitudinally in the distal direction without rotating, and with the radially-extending flange 13c on the head 13a of the drive rod 13 moving towards the first inner shoulder 14d of the body 14. In this partially-primed position, the shaft 13d of the drive rod 13 remains partially extended through the interior of the syringe 18 and, as shown, may begin to engage the syringe plunger stopper 18b within the syringe 18.

FIG. 3B is a perspective view of the proximal components of the dosing system 10 in a position at which a user has continued to apply the first longitudinal, distally-directed pressure to the button 11, this time with certain components of the system 10 in phantom so as to better illustrate the interior components therewithin, the collar 12 and the body 14 again being shown in phantom. As shown again here, the radially-extending protuberances 11d of the button 11 are moving through the first set of longitudinal slots 12g that extend longitudinally along the inner surface of the collar 12. As shown in FIG. 3B, the radially-extending flange 13c on the head 13a of the drive rod 13 is in contact with the first inner shoulder 14d of the body 14. This contact prevents a further distal movement of the drive rod 13.

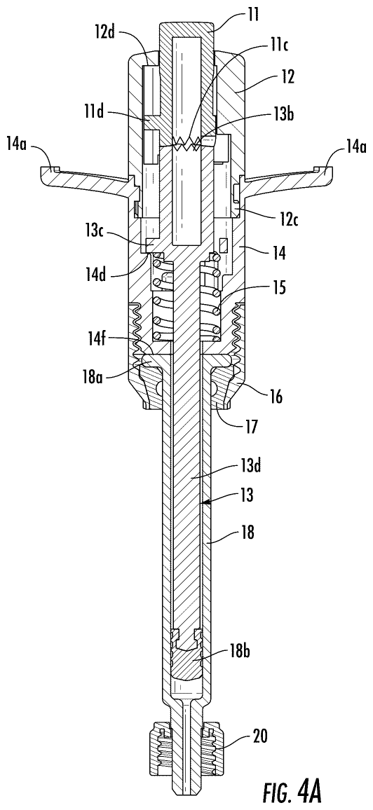

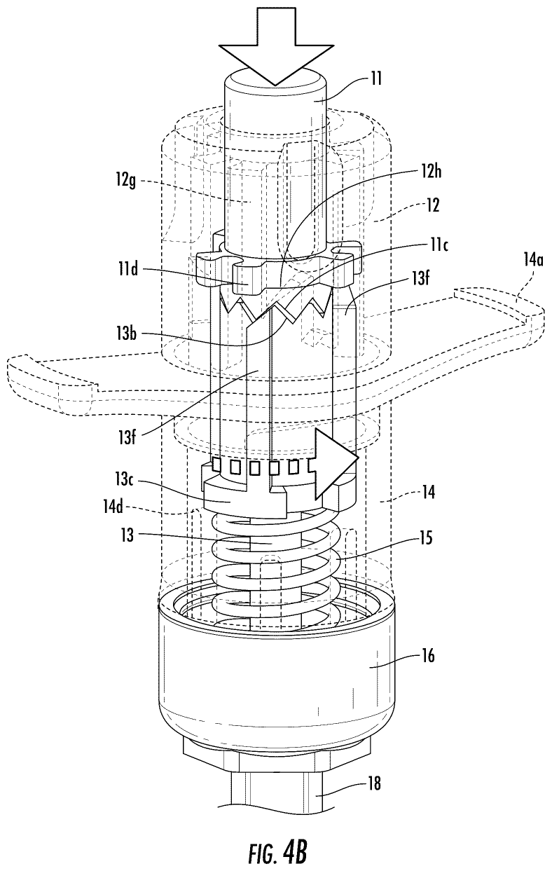

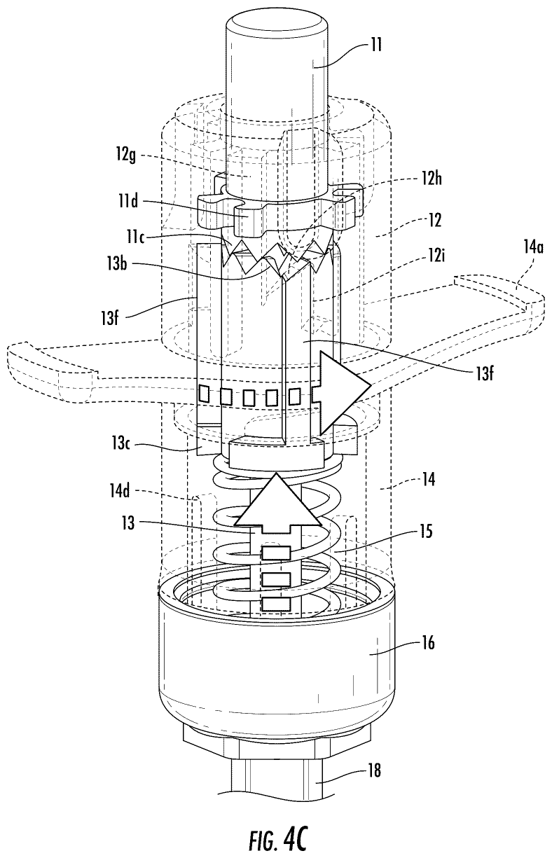

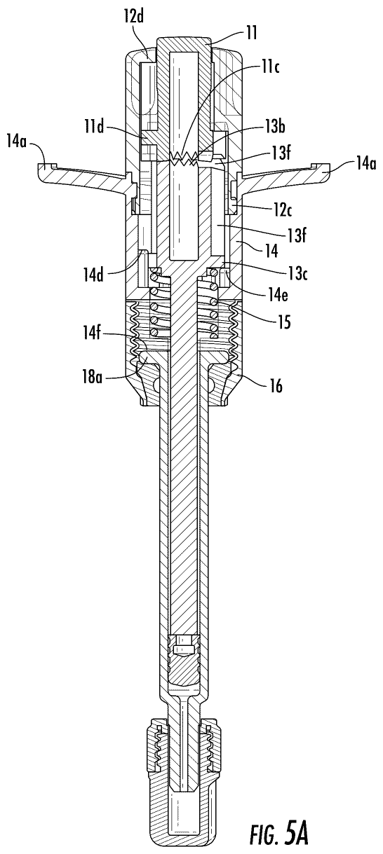

FIG. 4A is an assembled side cross-sectional view of the dosing system 10, in accordance with various embodiments, showing the components thereof in a fully assembled position, and showing the dosing system 10 in the same position than in FIG. 3B. As shown in FIG. 4A, the button 11 has moved further distally compared to FIG. 3A with the series of radially-extending protuberances 11d of the button 11 still further spaced apart from the radially-inwardly extending shoulder 12d of the collar 12. In this position, the force of the spring 15 has been still further overcome by the force applied by the user to the button 11, with the radially-extending protuberances 11d of the collar 11 travelling along and through the longitudinal slots 12g along the inner surface of the collar 12 until the radially-extending flange 13c on the head 13a of the drive rod 13 is in contact with the first inner shoulder 14d of the body 14. Also, in this position, the shaft 13d of the drive rod 13 extends through the interior of the syringe 18 and, as shown, further engages the syringe plunger stopper 18b within the syringe 18. In this position, the protrusions 13f of the drive rod 13 have cleared the point at which the longitudinal slots 12g intersect the first ramps 12h, the drive rod 13 is then no longer blocked in rotation. Advantageously, the distance that the drive rod 13 has travelled through the interior of the syringe 18 is a predetermined amount that has been determined to effectively purge any air or excess medication from the system 10.

FIG. 4B is a perspective view of the proximal components of the dosing system 10, with the collar 12 and the body 14 shown in phantom. As shown here, the radially-extending protuberances 11d of the button 11 have moved through the first set of longitudinal slots 12g and the protrusions 13f of the drive rod 13 have cleared the point at which the longitudinal slots 12g intersect the first ramps 12h, thereby enabling the drive rod 13 to rotate such that the proximally-extending teeth 13b at the proximal end of the drive rod 13 are free to slide along and engage with the plurality of distally-extending teeth 11c of the button 11. The protrusions 13f are now positioned distally relative to the first ramps 12h. As shown in FIG. 4B, the radially-extending flange 13c on the head 13a of the drive rod 13 is in contact with the first inner shoulder 14d of the body 14.

FIG. 4C is another perspective view of the proximal components of the dosing system 10 with the collar 12 and the body 14 again being shown in phantom. In this figure, with the user having released the first application of pressure on the button 11, the drive rod 13 has rotated sufficiently such that the protrusions 13f have fully slid proximally along the angled lengths of the first ramps 12h. The drive rod has thus achieved a first drive rod rotation. In doing so, the protrusions 13f contact the longitudinal-extending wall 12i on the inner surface of the collar 12 which acts as a hard stop to prevent further movement of the drive rod 13 (unless and until the user presses the button 11 a second time in order to dispense the dose of medication, which is described in additional detail below). As shown in FIG. 4C, because the ramp 12h is angled slightly proximally as it extends along the inside wall of the collar 12, by the time the protrusions 13f contact the hard stop of the longitudinal-extending wall 12i, the radially-extending flange 13c on the head 13a of the drive rod 13 has retracted slightly in a proximal direction away from the first inner shoulder 14d of the body 14, reflecting that the user has released pressure on the button 11. Moreover, after the first drive rod rotation, the radially-extending flange 13c on the head 13a of the drive rod 13 is no longer in alignment with the first inner shoulder 14d of the body 14 along the longitudinal direction. In this position, the dosing system 10 is fully primed.

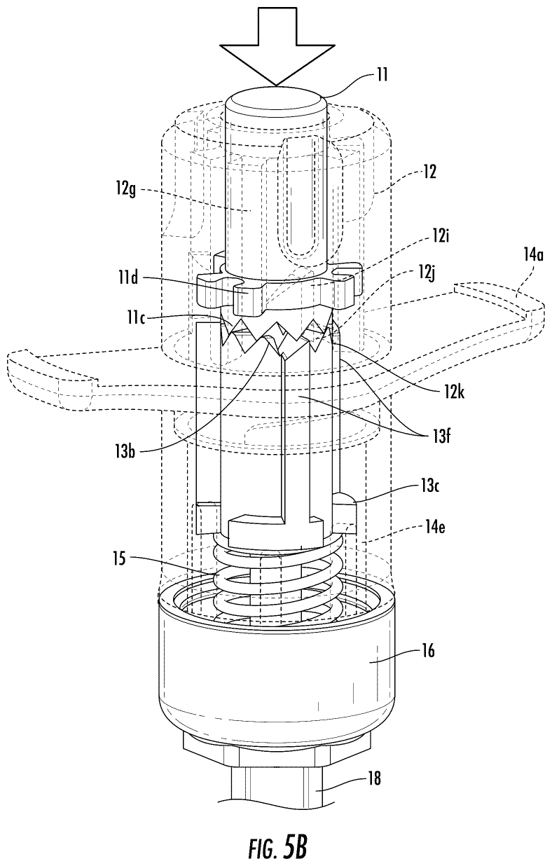

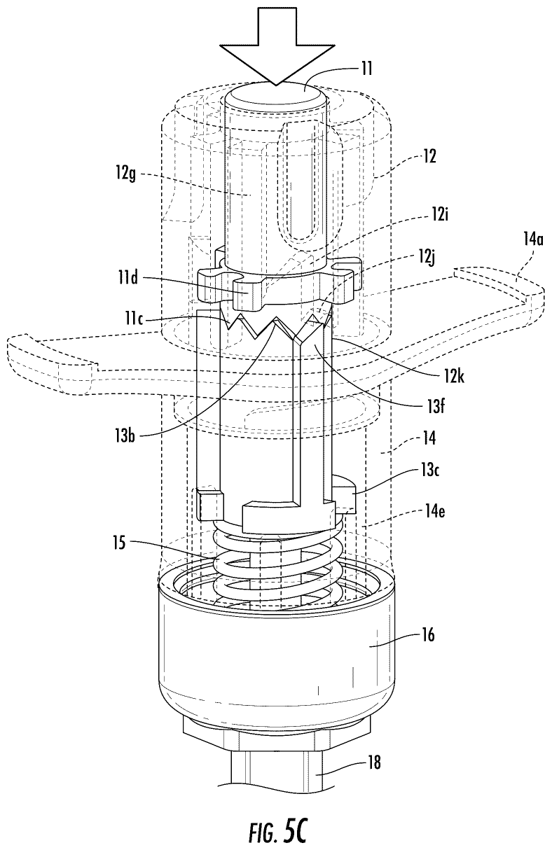

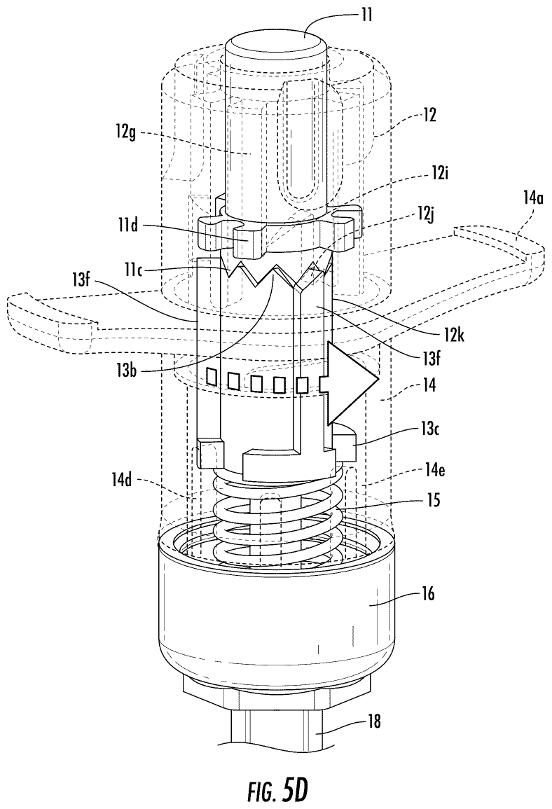

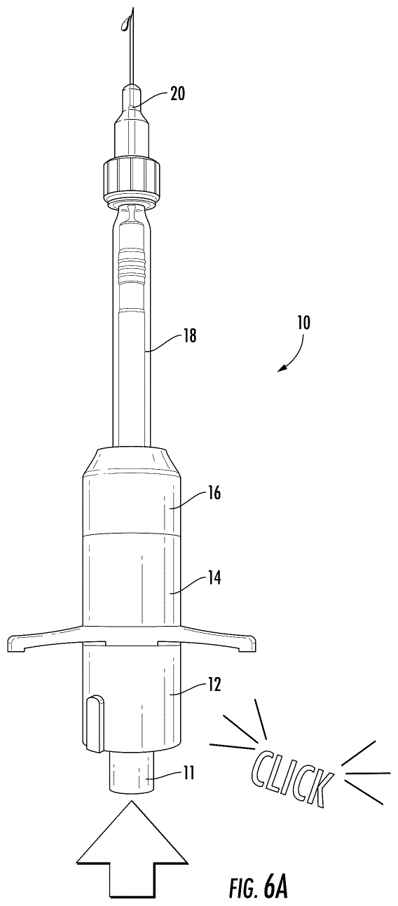

FIG. 5A is an assembled side cross-sectional view of the dosing system 10, in accordance with this embodiment, showing the components thereof in a fully assembled position, and showing the dosing system 10 in a position at which the user has pressed the button 11 a second time, e.g., applied a second longitudinal, distally-directed pressure to the button 11. This second longitudinal, distally-directed pressure to the button 11 functions to administer the dose of medication by the syringe 18, as will be set forth in additional detail below.

As shown in FIG. 5A, the button 11 has moved further distally, the plurality of distally-extending teeth 11c of the button 11 are offset from the plurality of proximally-extending teeth 13b at the proximal end of the drive rod 13. In this position, the force of the spring 15 has again been overcome by the second force applied by the user to the button 11, with the radially-extending protuberances 11d of the button 11 continuing to travel along and through the longitudinal slots 12g along the inner surface of the collar 12. Because the drive rod 13 was rotated, the radially-extending flange 13c on the head 13a of the drive rod 13 is no longer in alignment with the first inner shoulder 14d of the body 14 and thus the drive rod 13 can move distally past the position noted above at which the radially-extending flange 13c on the head 13a of the drive rod 13 contacted the first inner shoulder 14d of the body 14. Instead, the radially-extending flange 13c on the head 13a of the drive rod 13 is now free to travel still further distally until it is in contact with the second inner shoulder 14e of the body 14. In this position, the shaft 13d of the drive rod 13 is extended through the interior of the syringe 18 at a distal-most position and, as shown, the syringe plunger stopper 18b is moved distally at a distal-most position within the syringe 18 such that part of the medication located within the syringe 18 and positioned distally relative to the syringe plunger stopper 18a is dispensed out of the distal-most tip of the syringe 18. The dosing system 10 is in a dosed position. Advantageously, the distance that the drive rod 13 has travelled through the interior of the syringe 18 during the second pressure of the user on the button 11 is a predetermined amount that has been determined to dispense the precise dose of medication that is desired to be applied, with the position of the second inner shoulder 14e within the body 14 ensuring that the distance travelled is not longer than desired, as explained below. The distance between the first inner shoulder 14d and the second inner shoulder 14e corresponds to a dosing course of the drive rod 13.

FIG. 5B is a perspective view of the proximal components of the dosing system 10 in this same dosed position, with the collar 12 and the body 14 again being shown in phantom. As shown here, as just explained in connection with FIG. 5A, the radially-extending protuberances 11d of the button 11 have moved through the first set of longitudinal slots 12g and the protrusions 13f have distally cleared the point at which the longitudinally-extending walls 12i intersect the second ramps 12j. As shown in FIG. 5C, having reached this position, the drive rod 13 is again able to rotate such that the proximally-extending teeth 13b at the proximal end of the drive rod 13 slide along and engage with the plurality of distally-extending teeth 11c of the button 11. The protrusions 13f are now positioned distally relative to the second ramps 12j. The dosing system 10 is in a partially locked position. As shown in FIGS. 5B and 5C, the radially-extending flange 13c on the head 13a of the drive rod 13 is in contact with the second inner shoulder 14e of the body 14, which ensures that the correct dosing stroke is achieved without the user being able to inadvertently press the button 11 too far which could potentially cause an overdose of the medication to be applied.

As shown in FIG. 5D, the user having released the second application of pressure on the button 11, the drive rod 13 has rotated such that the protrusions 13f have fully slid along the lengths of the second ramps 12j. In doing so, the protrusions 13f contact the longitudinal-extending stop wall 12k on the inner surface of the collar 12 which acts as a hard stop to prevent further movement of the drive rod 13. As shown in FIG. 5D, the ramp 12j is angled slightly proximally as it extends along the inside wall of the collar 12. Thus, when the user releases the second application of pressure on the button 11, the spring 15 pushes the drive rod 13 proximally such that the drive rod 13 is retracted slightly, whereby the protrusions 13f contact the hard stop of the longitudinal-extending stop wall 12k and the radially-extending flange 13c on the head 13a of the drive rod 13 has moved in a proximal direction away from the second inner shoulder 14e of the body 14. In this position, the dosing system 10, in particular the drive rod 13, is now locked against further movement. The dosing system 10 is in a fully locked position.

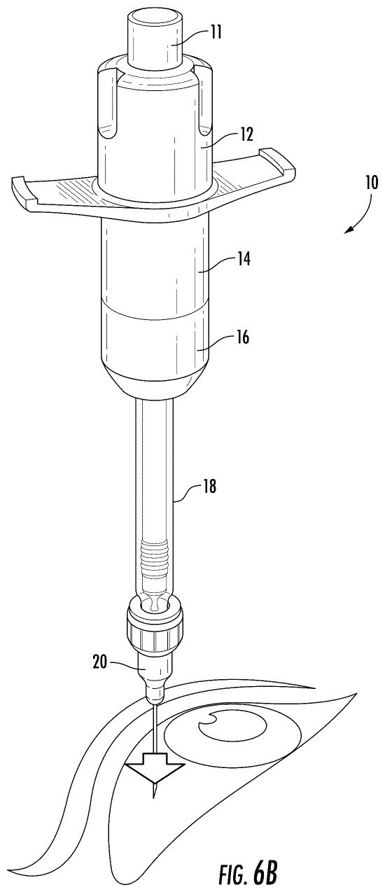

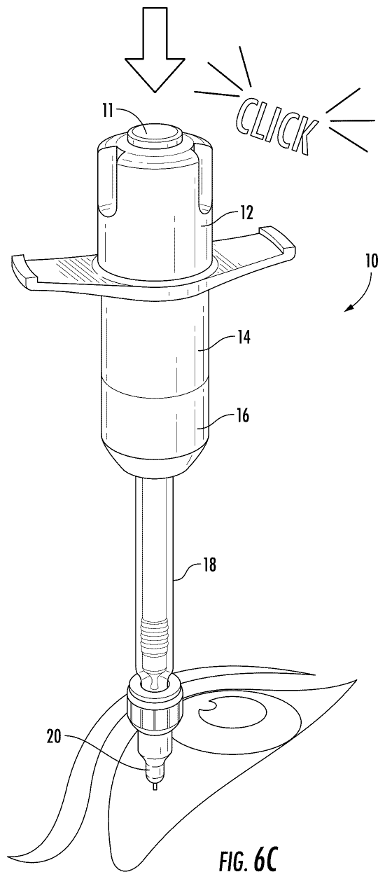

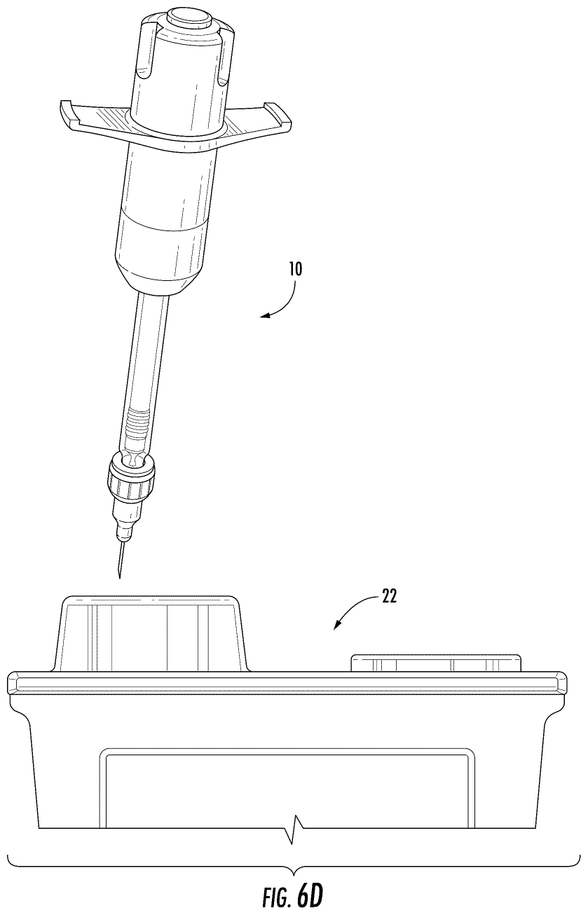

FIGS. 6A through 6D illustrate the operation of the dosing control system 10 set forth herein, in accordance with various embodiments. As set forth above, the dosing system described herein is ideally suited to accurately deliver of a precise dose of a small volume of medication, and it should be recognized that there is no limitation to the type of medication that can be dispensed or the type of application by which the medication can be dispensed. Using the example set forth above, namely that of accurately delivering of a precise dose of a small volume of ocular medication in an intraocular procedure, FIG. 6A illustrates a first step of the system's operation by which the user presses the button 11 a first time, e.g., by applying a first application of pressure to the button 11, advantageously hearing or tactile feeling a first click. Having done so, and thereafter releasing the first application of pressure on the button 11, the dosing system 10 is now primed, any excess air or medication being purged from the syringe 18 in the manner as set forth above.

FIG. 6B illustrates a second step of the system's operation by which the user positions the distal tip of the syringe 18 to or into the patient's eye. FIG. 6C illustrates a third step of the system's operation, by which the user presses the button 11 a second time, e.g., by applying a second application of pressure to the button 11. As mentioned above, the system 10 is advantageously configured such that the drive rod 13 is prevented from travelling distally beyond the distance at which it has supplied the correct dosage of medication. In addition, advantageously the user again hears or tactile feels a click, specifically a second click (as distinguished from the first priming click) which clicks upon completion of the button press so that the user has an audible or tactile indication that the correct dosage of medication has been fully dispensed. Finally, in FIG. 6D, the syringe 18 may be disposed of in a safe manner, e.g., by disposal into an appropriate sharps disposal container 22.

In the above embodiments, the first longitudinal, distally-directed pressure to the button 11 functions to help prime the syringe 18 and the second longitudinal, distally-directed pressure to the button 11 functions to dispense the dose of medication by the syringe 18. However, other embodiments are also envisioned in which the dosing system could also be used for a two-dose dispensing. In such an embodiment, each longitudinal, distally-directed pressure to the button 11 may function to dispense one of the two doses of medication by the syringe 18. Also, and as set forth above, while the above disclosure describes an embodiment of the dosing system in which two longitudinal, distally-directed pressures are applied to the button 11, other embodiments are also envisioned in which less than two (e.g. a single dosing step without priming or a single priming step without dosing,) or more than two (e.g., multiple priming and/or dosing steps) longitudinal, distally-directed pressures are applied to the button 11.

Again, this example of an intraocular operation is merely one example of many types of operations for which the dosing system 10 described herein, in accordance with various embodiments, may be employed. Mor specifically, there are no limitations in terms of the particular embodiments described in this application, which are intended as illustrations of various aspects only. Many modifications and variations can be made without departing from its spirit and scope, as will be apparent to those skilled in the art. Functionally equivalent methods and apparatuses, in addition to those enumerated herein, will be apparent to those skilled in the art from the foregoing descriptions. Such modifications and variations are intended to fall within the scope of the appended claims. Only the terms of the appended claims are intended to be limiting, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

What is claimed is:

1. A dosing system for a syringe, the dosing system comprising:
a housing configured to be attached to the syringe;
a first stop surface and a second stop surface;
a button extending from the housing and configured to be actuated by a user;
a drive rod extending at least partially through the housing and the syringe, a distal end of the drive rod configured to engage a plunger stopper within the syringe and a proximal end of the drive rod configured to be engaged by the button when the button is pressed by the user;
wherein, upon the user actuating the button a first time, the drive rod moves distally until it engages the first stop surface whereupon the drive rod rotates; and
wherein, upon the user actuating the button a second time, the rotated drive rod is able to move further distally until it engages the second stop surface.

2. The dosing system of claim 1, wherein the first stop surface is a first shoulder located on an inner surface of the housing, the first shoulder configured to be contacted by the drive rod when the user actuates the button the first time.

3. The dosing system of claim 2, wherein the second stop surface is a second shoulder located on the inner surface of the housing, the second shoulder located distally relative to the first shoulder and configured to be contacted by the drive rod when the user actuates the button the second time.

4. The dosing system of claim 1, further comprising a first anti-return surface configured to limit the drive rod proximal movement after the user releases the button a first time.

5. The dosing system of claim 4, wherein the first anti-return surface is a first ramp located on an inner surface of the housing, the first ramp configured to be contacted by the drive rod when the user, after having actuated the button the first time, releases the button the first time.

6. The dosing system of claim 1, wherein the drive rod includes a plurality of proximally-extending teeth and the button includes a plurality of distally-extending teeth, the drive rod being configured to rotate when the plurality of proximally-extending teeth engage the plurality of distally-extending teeth.

7. The dosing system of claim 5, wherein the drive rod comprises a radial protrusion, upon the user releasing the button the first time, the protrusion of the drive rod slide along the first ramp, the first ramp being angled proximally such that, upon the user releasing the button the first time, the drive rod moves proximally, while the protrusion is sliding along the first ramp, until the protrusion engage a first wall so as to be in a fully primed position.

8. The dosing system of claim 1, further comprising a second anti-return surface configured to limit the drive rod proximal movement after the user releases the button a second time.

9. The dosing system of claim 8, wherein the second anti-return surface is a second ramp located on an inner surface of the housing, the second ramp configured to be contacted by the drive rod when the user, after having actuated the button the second time, releases the button the second time.

10. The dosing system of claim 9, wherein the drive rod includes a plurality of proximally-extending teeth and a radial protrusion, and the button includes a plurality of distally-extending teeth, the drive rod is configured to further rotate when the plurality of proximally-extending teeth engage the plurality of distally-extending teeth, such that, upon the user releasing the button the second time, the protrusion slides along the second ramp, and wherein the second ramp is angled proximally such that, upon the user releasing the button the second time, the drive rod moves proximally, while the protrusion is sliding along the second ramp, until the protrusion engages a second wall so as to be in a locked position.

11. The dosing system of claim 1, further comprising a biasing member configured to move the drive rod proximally in the absence of force exerted by the user on the button during either the first and second actuations of the button.

12. The dosing system of claim 1, wherein the button has a protuberance cooperating with a slot of the housing so as to enable the button to move longitudinally and prevent rotation of the button when the user actuates the button.

13. The dosing system of claim 1, wherein the system includes an indicator providing information to the user on the position of the drive rod, and wherein the indicator is one or a combination of a tactile indicator that enables the user to feel at least one of when the drive rod has moved distally and when the drive rod has moved further distally and an audible indicator that provides an audible indication to the user at least one of when the drive rod has moved distally and when the drive rod has moved further distally and a visual indicator that provides a visual indication to the user at least one of when the drive rod has moved distally and when the drive rod has moved further distally.

14. The dosing system of claim 1, wherein the syringe is a pre-filled syringe.

15. The dosing system of claim 1, wherein the user actuating the button the first time corresponds to a priming step, and wherein the user actuating the button the second time corresponds to a dosing step.

16. A method for dispending a dose of medication from a syringe, the method comprising:
providing a dosing system that includes a housing configured to be attached to the syringe a first stop surface and a second stop surface, a button extending from the housing and configured to be actuated by a user, and a drive rod extending at least partially through each of the housing and the syringe;
pressing the button a first time such that the drive rod moves distally until it engages the first stop surface;
upon engaging the first stop surface, rotating the drive rod a first time;
releasing the button a first time such that the drive rod moves proximally until it engages a first anti-return surface;
pressing the button a second time so as to move the drive rod still further distally until it engages the second stop surface;
upon engaging the first stop surface, rotating the drive rod a second time; and
releasing the button a second time such that the drive rod again moves proximally until it engages a second anti-return surface.

17. A dosing system for a syringe, the dosing system comprising:
a housing configured to be attached to the syringe;
a first stop surface and a first stop wall;
a button extending from the housing and configured to be actuated by a user;
a rotatable drive rod extending at least partially through the housing and the syringe, a distal end of the drive rod configured to engage a plunger stopper within the syringe and the proximal end of the drive rod configured to be engaged by the button when the button is pressed by the user;
wherein, upon the user pressing the button a first time, the drive rod moves distally until it engages the first stop surface, and
wherein, upon the user releasing the button a first time, the drive rod rotates a first time until it contacts the first stop wall.

18. The dosing system of claim 17, wherein the user pressing the button the first time corresponds to either a priming step or a dosing step.

19. The dosing system of claim 17, wherein the dosing system comprises a second stop surface and is configured such that, when the user presses the button a second time, the rotated drive rod is free to move distally past the first stop surface until it engages the second stop surface.

20. The dosing system of claim 19, wherein the user pressing the button the second time corresponds to a dosing step.

21. The dosing system of claim 19, wherein the dosing system comprises a second stop wall and is configured such that, when the user releases the button a second time, the drive rod rotates a second time until it contacts the second stop wall.
